(19) United States
(12) Patent Application Publication
(10) Pub. No.: US 2011/0224312 A1
(43) Pub. Date: Sep. 15, 2011

(54) ENGINEERING POROUS PARTICLES OF WATER SOLUBLE THERAPEUTICS FOR PRESSURIZED METERED-DOSE INHALER FORMULATIONS

(75) Inventors: Sandro R.P. Da Rocha, Troy, MI (US); Libo Wu, Detroit, MI (US)

(73) Assignee: WAYNE STATE UNIVERSITY, DETROIT, MI (US)

(21) Appl. No.: 13/129,803

(22) PCT Filed: Nov. 17, 2009
(86) PCT No.: PCT/US09/64864

§ 371 Date: May 17, 2011

Related U.S. Application Data
(60) Provisional application No. 61/199,518, filed on Nov. 17, 2008.
(60) Provisional application No. 61/199,519, filed on Nov. 17, 2008.

(51) Int. Cl. A61K 47/24

(52) U.S. Cl. 514/785

(57) ABSTRACT

The subject matter disclosed herein relates to methods for engineering porous particles of water soluble therapeutics with enhanced physical stability and aerosol characteristics in hydrofluoroalkane (HFA)-based pressurized metered-dose inhaler (pMDI) formulations.

### STATEMENT OF GOVERNMENT INTEREST

This work was supported in part by National Science Foundation grant number CBET 0553537. The government has certain rights to the invention disclosed herein.

### FIELD

The subject matter disclosed herein relates to new methods for engineering porous particles of water soluble therapeutics with enhanced physical stability and aerosol characteristics in hydrofluoroalkane (HFA)-based pressurized metered-dose inhaler (pMDI) formulations.

### BACKGROUND

Pressurized metered-dose inhalers (pMDIs) are the least inexpensive, and the most widely used devices for pulmonary drug delivery.(1, 2) The propellant is the major constituent of pMDIs, where the active ingredients are either solubilized (solution formulations) or dispersed (suspension formulations).(3) pMDIs may also contain non-active excipients such as cosolvents, amphiphiles and flavors.(4) Dispersion-based formulations account for approximately half of the commercially available pMDIs, and have certain advantages over solution-based formulations such as the possibility of delivering higher dosages, and improved chemical stability of the therapeutic ingredients.(5, 6) Suspensions can be further classified with respect to the nature (solid or liquid) of the dispersed phase.(5, 7) The therapeutic molecules can be directly dispersed in the propellant in the solid form, as in micronized drug crystals or other particle-based dispersions—these represent current commercial suspension formulations.(5) Alternatively, it has been proposed that the active ingredient may be solubilized within a fluid phase dispersed in a liquid propellant.(8-13)

With the banning of the ozone depleting chlorofluorocarbon (CFCs),(14) alternative propellants have emerged. Hydrofluoroalkanes (HFAs) have been selected for use in pMDIs as they are biocompatible and environmentally acceptable.(14, 15) HFAs also have certain physical properties which are similar to CFCs, including density and vapor pressure, which to some extent facilitated the reformulation of pMDIs (in terms of the hardware design). However, the excipients typically used in FDA-approved, CFC-based pMDI formulations are generally not compatible with HFAs due to the differences in solvation forces, as HFAs are significantly more polar than CFCs.(4) Ethanol is, therefore, usually employed to enhance the solubility of excipients in HFAs.(16) However, the presence of co-solvents in the formulation may have undesirable effects, as for example to enhance the solubility of the active drug ingredients resulting in reduced chemical stability,(6) or to decrease the vapor pressure of the propellant mixture, thus affecting the aerosol performance.(16)

Many alternative pMDI formulations have been proposed as an attempt to address the reformulations issues that have affected the transition to HFA-based pMDIs. In the case of Proventil® HFA (Schering-Plough Corporation), as an example, the active ingredient (salbutamol base) was replaced by its salt, which has lower solubility in ethanol, an excipient used in that formulation. Changing the chemistry of the drug ingredient, however, is not always feasible. One potential alternative is the development of novel HFA-philic excipients that have high solubility in propellant HFAs, and thus do not require the use of ethanol in the formulation.(17-19) Within that context, recent investigations addressing solvation in HFAs have been relevant.(7, 12, 16, 20-23) Combined microscopic computational and experimental approaches have been employed to quantify HFA-philicity for several pharmaceutically relevant chemistries.(20, 21, 23) Such fundamental knowledge on solvation has in turn been used to design novel amphiphiles capable of stabilizing dispersions in the low dielectric HFAs.(17, 21) Those results have also helped in the development of novel particle engineering approaches that allow for the direct modification of the surface chemistry of the drug particles, thus enhancing the physical stability in the propellant and improving the characteristics of the corresponding aerosol formulations.(24, 25)

Engineering approaches that involve the modification of the chemistry of the particle surface have certain advantages compared to the stabilization of suspensions with surfactants in solution.(26, 27) Surfactant-stabilized suspensions may have a significant amount of free-amphiphile in solution, and thus potentially enhanced toxicity. Moreover, while the stabilizing moiety may be universal, there may be a need to tailor the anchoring tail (one that interacts with the drug) and the surfactant balance to the particular therapeutic molecule of interest.(17, 21) However, methodologies that use the direct modification of the particle surface still require excipients that are well solvated by the propellant, which may not be one of those employed in FDA-approved pMDIs.(17) Current particle surface-modification technologies also have some limitations in terms of the types of drugs that can be formulated.(25) Within this context, the ability to impart physical stability to the drug formulation by simply altering the surface morphology of the particle is of great interest. One development in this area the concept of porous particles.(28-32) Porous particles show enhanced stability in the propellant due to (i) the tact that the propellant is able to penetrate into the particle, thus functioning as a density matching with the liquid propellant, which in turn reduces the effect of gravitational fields; and (ii) the reduced (number) density and the reduced available surface contact area in porous particles, which help reduce the van der Waals attractive interactions, and thus minimize cohesive forces.(33, 34)

There remains a need in the art however for a novel methodologies for engineering porous particles of water soluble drugs with enhanced physical stability in propellant HFAs, and improved aerosol characteristics of the corresponding pMDI formulations.

### SUMMARY

Embodiments disclosed herein provide new methodologies for engineering porous particles of water-soluble active ingredients with enhanced physical stability and aerosol characteristics in HFA propellants for use in pMDI formulations.

Certain embodiments disclosed herein relate to methods of preparing a porous particle comprising at least one water soluble therapeutic, wherein said methods employ a modified emulsification-diffusion technique. In certain embodiments, the porous particle can be employed in a pressurized metered-dose inhaler formulation. In other certain embodiments, the inhaler formulation comprises hydrofluoroalkane gas.

Certain embodiments relate to methods of preparing a porous particle comprising at least one water soluble therapeutic, wherein said method employs a modified emulsification-diffusion technique comprising the preparation of a reverse aqueous emulsion comprising a solubilized drug and negatively charged lecithin particles, stabilized by an ionic surfactant adsorbed at the oil-water interface. In certain embodiments, the method for preparing a porous particle further comprises washing away lecithin particles to create a porous morphology.

In certain embodiments the lecithin particles are washed away using a interfacially active species having the same charge as the lecithin particles.

### BRIEF DESCRIPTION OF THE FIGURES

Exemplary embodiments are illustrated in referenced figures. It is intended that the embodiments and figures disclosed herein are to be considered illustrative rather than restrictive.

FIG. 1. FIG. 1 is a schematic illustration of the methodology for engineering porous particles using a modified version of the emulsification-diffusion technique. (a) reverse aqueous emulsion containing the solubilized drug and the negatively charged lecithin particles (small black circles), stabilized by an ionic (same charge as the dispersed particles) surfactant (represented by the two tails tethered to the head-group) adsorbed at the oil-water interface; (b) amorphous drug particle (large circle) formed after the diffusion of water into the organic phase, as the emulsion is contacted with excess organic phase. Lecithin particles (smaller black circles within the drug particle) are trapped within the drug particle; (c) lecithin particles are dissolved into the organic phase, giving rise to pores (small white circles) in the drug particle (larger circle). Some lecithin particles (small dark circles) may remain trapped.

FIGS. 2A-F. FIGS. 2A-F are a SEM of SS and THS particles prepared with different surfactants at fixed lecithin (20 mg·ml⁻¹) and surfactant (2.5 mg·ml⁻¹) concentration. SS particles formed with (A) oleic acid, (B) Pluronic® L81 (“L81”) (BASF), and (C) AOT—Inset: TEM image of the porous SS particle. THS particles formed with (D) oleic acid, (E) L81, and (F) AOT—Inset: TEM image of the porous THS particles.

FIG. 3. FIG. 3 shows the tension of the water-ethyl acetate interface in presence of oleic acid and AOT, at 298 K.

FIGS. 4A-F. FIGS. 4A-F show SS particles prepared (1) at fixed lecithin concentration of 20 mg·ml⁻¹, with varying AOT concentration of (A) 0, (B) 0.5, (D) 1.5, and (E) 2.5 mg·ml⁻¹; (2) at low level (fixed) AOT concentration of 0.5 mg·ml⁻¹, with varying lecithin concentration of (B) 20 and (C) 35 mg·ml⁻¹; (3) at higher level (fixed) of AOT concentration of 2.5 mg·ml⁻¹, with lecithin concentration of (F)₅; (E) 20 mg·ml⁻¹.

FIGS. 5A-D. FIGS. 5A-D show the dispersion stability of SS and THS particles in HFA134a and HFA227. (A) Solid SS particles prepared by emulsification-diffusion without using AOT and lecithin; (B) Porous SS particles prepared with AOT concentration of 2.5 mg·ml⁻¹ and lecithin of 20 mg·ml⁻¹; (C) Solid THS particles from emulsification-diffusion without using AOT and lecithin; (D) Porous THS particles prepared with AOT concentration of 2.5 mg·ml⁻¹ and lecithin of 20 mg·ml⁻¹. FIG. 6. Aerodynamic particle size distribution of Ventolin® HFA (GlaxoSmithKilne), solid SS and porous SS formulations in HFA134a, at 2 mg·ml⁻¹. (a) without spacer; (b) with spacer. The porous particles were prepared with an AOT concentration of 2.5 mg·ml⁻¹ and 20 mg·ml⁻¹ lecithin.

FIGS. 6A and B. FIGS. 6A and B consist of bar graphs showing aerodynamic particle size distribution of Ventolin® HFA, solid SS and porous SS formulations in HFA134a, at 2 mg·ml⁻¹. (A) without spacer; (B) with spacer. The porous particles were prepared with an AOT concentration of 2.5 mg·ml⁻¹ and 20 mg·ml⁻¹ lecithin.

### DETAILED DESCRIPTION

This disclosure is directed to nanoparticle engineering, particle-surface engineering (particularly porous particle engineering) and stabilization of suspension-based formulations in inhaled therapies, including, without limitation, gas propellants, and in one embodiment hydrofluoroalkane (HFA) gas.

Methodologies for generating porous structures are described herein. A representative strategy used in this work to engineer porous particles is schematically illustrated in FIG. 1. The methodology consists of using a modified emulsification-diffusion technique to generate amorphous drug particles containing encapsulated lecithin nanoparticles, which may be later extracted out (the nanoparticles), thus generating a porous structure. An active drug ingredient can first be dissolved in an aqueous dispersion containing nanoparticles. As a non-limiting example, lecithin particles can be used. The aqueous dispersion containing the drug (solubilized) can then be emulsified in an organic solvent containing a surfactant, to obtain a water-in-oil (W/O) emulsion. The organic phase can be selected so as to have high water loading capacity (large solubility of water). As a non-limiting example, ethyl acetate (Ac) can be used. The emulsion can then be diluted in a larger volume of the organic solvent. During this step, water diffuses out from within the droplets and into the organic phase. During the diffusion process, the negatively charged lecithin particles can remain within the water droplets that make up the dispersed phase of the emulsion. This can be accomplished by without limitation using an ionic surfactant with the same charge as lecithin (anionic surfactant). The trapped lecithin particles can be subsequently removed by washing (with, in one non-limiting embodiment, hexane), thus leading to a porous morphology. The porosity of the particles can be controlled by, one or more of without limitating, adjusting the amount of surfactant at the interface, adjusting the concentration and size of lecithin particles, and adjusting the size of the drug particles by without limitation varying the amount of mechanical energy used to form the initial reverse emulsion.

The effect of preparation parameters on the particle morphology was investigated and is described in detail below. The applicability of the proposed methodology for Salbutamol Sulfate (SS) and Terbutaline Hemisulfate (THS) as model drugs is demonstrated, but the approach is applicable/transferable to a wide range of water-soluble drugs. The effect of preparation parameters on the morphology of the porous drug particles was also evaluated. The resulting physical (bulk) stability of the dispersions in 1,1,1,2-tetrafluoroethane (HFA134a) and 1,1,1,2,3,3,3-heptafluoropropane (HFA227), and the performance of the corresponding aerosols were examined, and directly compared to non-porous particles and to existing commercial formulations.

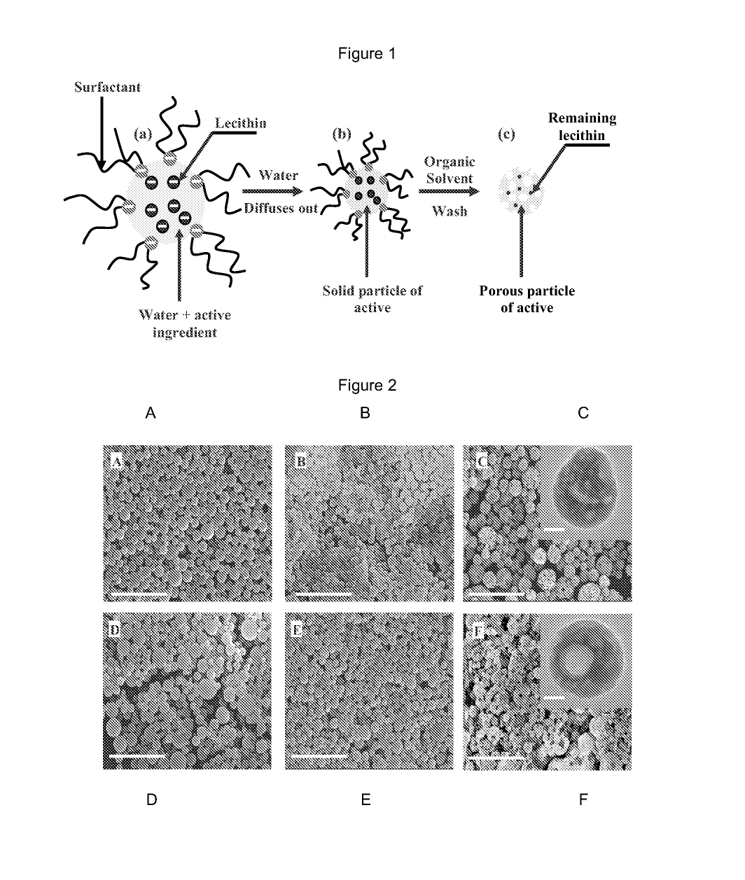

The effect of surfactant types was also evaluated. Ionic and non-ionic surfactants can be used to stabilize the W/Ac emulsions. The morphology of the various particles is shown in FIG. 2. Porous structures were only obtained when AOT was used (FIGS. 2C and E and TEM insets). This is true for both SS and THS. Solid spherical particles can be formed when the emulsions are stabilized with L81 and oleic acid (FIGS. 2A, B, D and E). For L81, the resulting solid morphology can be rationalized based on the fact that L81 is a nonionic surfactant, thus providing for a weak barrier for the movement of the negatively charged lecithin particles that would tend to migrate out of the water droplets during the diffusion process—when the emulsion is diluted to large volumes of ethyl acetate. It is worth noticing that lecithin particles are dispersible in ethyl acetate saturated with water, and if no barrier is present, they may freely escape the interior of the emulsion droplet phase along with the water that diffuses out.

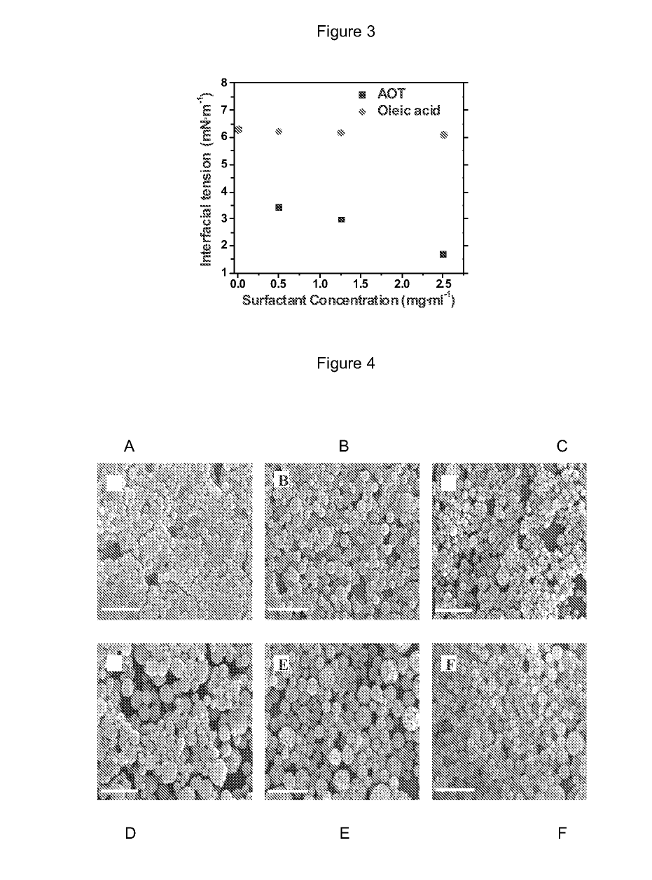

As oleic acid is negatively charged, in principle it should be able to restrain the lecithin nanoparticles from leaving the emulsion droplet phase. To elucidate the phenomenon, the activity of oleic acid at the water-ethyl acetate system was determined, and the results are shown in FIG. 3.

It was observed that the tension of the water-ethyl acetate interface remained almost unchanged with the introduction of oleic acid. In contrast, AOT was very active at the interface, significantly reducing the interfacial tension. These results, along with the observation that porous particles were observed only when the emulsions were stabilized with AOT, suggest that the mechanism of retention of the negatively charged lecithin nanoparticles within the emulsion phase is electrostatic repulsion; i.e., the anionic surfactant AOT, which is capable of strongly adsorbing at the water-ethyl acetate interface, prevents the diffusion of the lecithin nanoparticles along with water during the diffusion step of the emulsification-diffusion method. On the other hand, oleic acid did not prevent the diffusion of lecithin particles because of its low activity at the interface, and L81® because it is not charged.

The effect of AOT and lecithin concentration were analyzed. Once the mechanism for the generation of the porous structures was established, the effect of the concentration of lecithin and AOT on the morphology of the particles was investigated. The results are summarized in FIG. 4.

The SEM images of SS particles prepared at fixed concentration of lecithin nanoparticles at 0.5 mg·ml⁻¹, and with increasing concentrations of AOT of 0, 0.5, 1.5 and 2.5 mg·ml⁻¹ are shown in FIGS. 4A, B, D and E, respectively. It can be observed from FIG. 4A that without AOT, as expected, solid particles are formed. It can also be qualitatively observed that the porosity of the SS particles is directly proportional to the concentration of AOT. Higher bulk concentrations of AOT should translate, up to its solubility limit or critical aggregation concentration, to larger adsorbed amounts at the interface. An increase in interfacial concentration should in turn help retain the lecithin nanoparticles confined within the droplet phase. The end result is an increase in porosity of the drug particles. At fixed AOT concentration, the porosity can be seen to be directly related to the concentration of lecithin nanoparticles—compare FIGS. 4B and C, and FIGS. 4E and F. An increase in concentration of lecithin nanoparticles, either by being retaining at larger fractions (as observed when the concentration of AOT is increased), or by increasing the initial amount of lecithin nanoparticles, should result in larger fractions of lecithin retained within the solid particle, and thus a more porous structure as the lecithin is removed (washed out). The ability to control particle porosity is one of the advantages of the proposed methodology. It is expected that the final size of the porous particles can be easily controlled simply by changing the size of the initial emulsion droplet size (mechanical energy input). Such flexibility allows one to fine tune the aerosol characteristics of the formulation.

The chemical composition of the porous particles was also analyzed. The overall concentration of active ingredient in the SS and THS particles was analyzed by UV-vis spectroscopy. The content of SS in the porous particles varied from 78 to 91 wt %, depending on the overall concentration of AOT and lecithin used in the preparation process. The amount of AOT residue in the SS particles was negligible, and was found to be undetectable by UV-vis. The particle, therefore, can be made of the active excipient and remaining lecithin, which is an excipient used in FDA-approved pMDIs.(4)

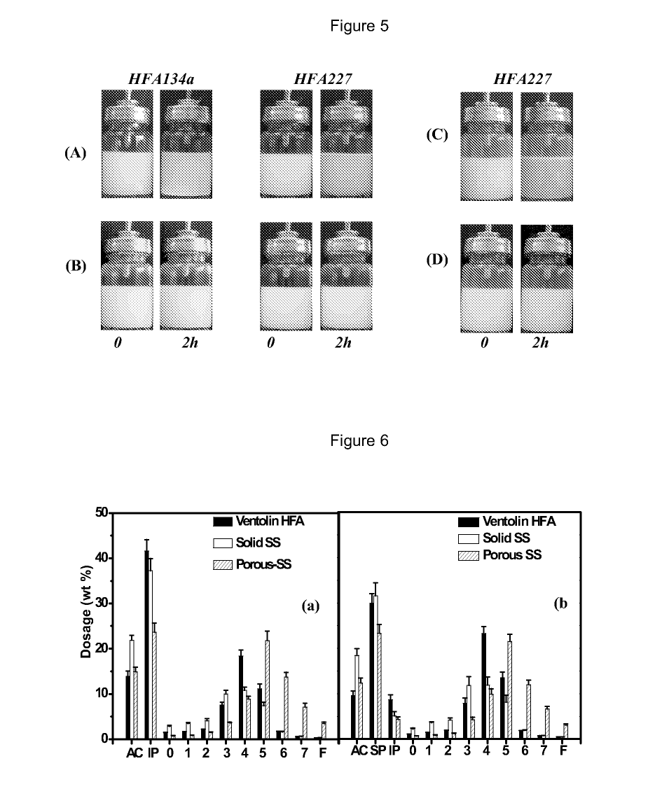

The physical stability of the formulations in HFA propellants was studied. Sedimentation rate experiments of the porous SS particles prepared with AOT concentration of 2.5 mg·ml⁻¹ and 20 mg·ml⁻¹ of lecithin were performed in HFA134a and HFA227 at 298 K and saturation pressure of the propellant. Results for porous THS prepared under the same conditions in HFA227 are also presented, to further demonstrate and elaborate on the extent of the applicability of the method. The results are summarized in FIG. 5. Colloidal stability results for solid SS/THS particles prepared without AOT and lecithin are also reported, and serve as the baseline.

The physical stability results shown in FIG. 5A indicate that solid SS spheres obtained from emulsification-diffusion have poor stability in the hydrofluoroalkane propellants HFA134a and HFA227. Creaming of the particles in HFA227 or sedimentation in HFA134a started taking place immediately after mechanical input (used for dispersing the particles) was stopped. On the other hand, excellent stability was achieved with the porous SS particles in both HFA227 and HFA134a, with very long term stability, as shown in FIG. 5B. Such dramatic improvement in physical stability agrees well with the suspension behavior of hollow porous particles described in previous work.(33, 34) A better dispersion can be expected due to a decrease in the density difference between the particle (with the propellant) and the bulk propellant, and also due to reduced van der Waals attractive forces between the particles. Physical stability results for THS in HFA227 were qualitatively similar to that for SS, as seen in FIGS. 5C and D.

Aerosol performance was analyzed as follows. Anderson cascade impactor (ACI) was utilized to characterize the aerosols of both the solid and porous particle-based SS pMDI formulations in HFA134a. The effect of a spacer was also investigated. The results were benchmarked against a commercial formulation of SS, Ventolin® HFA, which was previously tested in our laboratories.(24, 25) Aerosol results for porous THS in HFA227 are also shown, again to demonstrate the potential scope of the methodology to formulate other water soluble drugs. The ACI results are summarized Table 1, and plotted as wt. % in FIGS. 6A and B. The amount retained at each stage of the ACI is reported as dosage percentage of the total amount of drug delivered from the pMDI. Plotting the results in terms of % allows one to directly compare the different formulations.

TABLE I
Aerodynamic properties of porous SS and THS formulations in HFA
as probed by the ACI test (10 × actuation dose)

| | Porous SS | | |
|---|---|---|---|
| | HFA134a (n = 3) | | Porous THS |
| | No | With | HFA227 (n = 3) |
| Stages | spacer | spacer | No spacer |
| AC | 137.2 ± 11.2 | 114.9 ± 12.5 | 117.5 ± 9.8 |
| SP | N/A | 205.4 ± 18.6 | N/A |
| IP | 212.5 ± 15.6 | 37.9 ± 5.3 | 223.7 ± 17.7 |
| Stage 0 | 6.6 ± 1.3 | 5.9 ± 1.0 | 8.7 ± 1.5 |
| (9.0-10.0 μm) | | | |
| Stage 1 | 8.5 ± 0.9 | 7.4 ± 1.2 | 7.5 ± 1.0 |
| (5.8-9.0 μm) | | | |
| Stage 2 | 12.5 ± 2.0 | 11.0 ± 2.1 | 14.5 ± 1.4 |
| (4.7-5.8 μm) | | | |
| Stage 3 | 33.5 ± 3.4 | 37.6 ± 4.7 | 36.5 ± 2.8 |
| (3.3-4.7 μm) | | | |
| Stage 4 | 80.5 ± 6.2 | 89.6 ± 7.9 | 77.6 ± 9.0 |
| (2.1-3.3 μm) | | | |
| Stage 5 | 197.4 ± 16.9 | 198.7 ± 15.5 | 181.8 ± 14.6 |
| (1.1-2.1 μm) | | | |
| Stage 6 | 124.5 ± 9.6 | 105.8 ± 10.0 | 113.6 ± 8.8 |
| (0.7-1.1 μm) | | | |
| Stage 7 | 64.6 ± 4.5 | 59.6 ± 6.2 | 63.5 ± 3.7 |
| (0.4-0.7 μm) | | | |
| Filter | 24.6 ± 3.4 | 27.5 ± 2.5 | 20.8 ± 2.1 |
| (0-0.4 μm) | | | |
| FPF (%) | 68.6 ± 2.1 | 89.0 ± 2.4 | 66.5 ± 1.4 |
| MMAD (μm) | 1.4 ± 0.1 | 1.4 ± 0.1 | 1.5 ± 0.1 |
| GSD (μm) | 2.1 ± 0.2 | 2.1 ± 0.1 | 2.1 ± 0.2 |

Both the porous SS and THS particles were prepared with AOT concentration of 2.5 mg · ml⁻¹ and 20 mg · ml⁻¹ lecithin.
AC, IP, SP and F refer to actuator & valve stem, induction port, spacer and filter respectively.
n is the number of repeat experiments.
Deviation obtained from three independent measurements.

The aerosol performance of the porous SS formulation is significantly improved relative to both the commercial formulation and the solid SS particles (formed using the emulsification-diffusion technique).(24, 25) The fine particle fraction (FPF) for the porous formulation was 68.6%, compared to only 45.9% for Ventolin® HFA and 39.1% for solid SS.(25) The amount of drug retained in stages 5, 6 and 7 is particularly improved in the porous particle formulation, indicating the potential of the proposed particle engineering methodology to develop formulation for the enhanced delivery to the deep lungs. Further optimization of the proposed formulation, in terms of without limitation the particle size or porosity, and the hardware used, may be possible relative to that of the commercial formulation. These results are comparable to the aerosol performance of porous particles prepared by spray drying.(33)

The aerosol characteristics for the porous SS formulation in the presence of a spacer is further improved, with an FPF of 89.0%. The porous formulation produced aerosols with smaller MMAD (1.4 μm) than both commercial and solid SS formulations. The formulation with porous THS in HFA227 showed similar behavior as that for porous SS, with a FPF of 66.5% (no spacer), as shown in Table 1. These results reveal a correlation between the enhanced physical stability for the formulations containing the porous particles, and an improved aerosol characteristic of the corresponding formulations.

This disclosure provides a methodology for engineering porous particles of water-soluble active ingredients with enhanced physical stability and aerosol characteristics in HFA propellants for use in pMDI formulations. A modified emulsification-diffusion technique can be utilized to engineer the porous particles. The porous morphology can be generated by washing away lecithin nanoparticles, which are trapped within the drug matrix with the help of an interfacially active species (AOT) that has the same charge as lecithin particles.

The applicability of the methodology to polar active ingredients was tested with SS and THS. It was demonstrated that the porosity of the particles can be controlled by varying the concentration of lecithin particles and of AOT. The size of the porous particles can also be expected to be easily controlled by varying the amount of energy input during the emulsion formation step. Long term physical stability of porous SS and THS in HFAs was observed. ACI tests demonstrated a significantly improvement in FPF for porous SS formulation when benchmarked against Ventolin® HFA, a commercial SS formulation, reaching 89% with the help of a spacer. Similar results were observed for THS, suggesting this to be a generally applicable methodology to water solubilize active ingredients. The proposed methodology offers significant promise as the final particle can be composed of up to 90% of the active ingredient, and the remaining concentration can be made up of lecithin, an excipient commonly used in FDA-approved pMDIs.

### EXAMPLES

The following materials and methods were used throughout the Examples herein. Pharma grade hydrofluoroalkanes (HFA134a and HFA227, assay >99.99%) were obtained from Solvay Fluor and Derivate GmbH & Co. (Hannover—Germany). 2H,3H-perfluoropentane (HPFP) was obtained from SynQuest Labs Inc, with a purity of 98%. Salbutamol sulfate (SS) was obtained from Spectrum Chemicals. Terbutaline hemisulfate (THS) salt was from Sigma. The Pluronic® L81 (EO₃PO₄₃EO₃, where EO and PO stand for ethylene oxide and propylene oxide, and the subscripts are the average number of repeat units) surfactant was donated by BASF, and used as received.

Oleic acid (99%) and lecithin (refined) were from Aldrich and Alfa Aesar, respectively. Bis-(2-ethylhexyl)sodium sulfosuccinate (AOT) was from Sigma. Deionized water (NANOpure® DIAMOND™ UV ultrapure water system: Barnstead International), with a resistivity of 18.2 MΩ·cm and surface tension of 73.8 mN·m⁻¹ at 296 K, was used in all experiments. All the other organic solvents were obtained from Fisher Chemicals and were of analytical grade.

### Example 1

### Particle Engineering

Solid drug particles were prepared by a modified emulsification-diffusion technique. Initially, 25 mg of the drug of interest was dissolved in 0.8 ml of water. The aqueous solution containing the drug was then emulsified in 19 ml of ethyl acetate using a sonication bath (VWR, P250D, set to 180 W) at 303 K. A water-in-oil (W/O) emulsion was thus obtained. The emulsion was subsequently added to a large volume (150 ml) of ethyl acetate. Spherical drug particles are formed as water that makes the dispersed emulsion phase diffuses out into ethyl acetate.

The particles were collected by centrifugation. Porous particles were also prepared by emulsification diffusion, using a procedure similar to that described above. In the case of porous particles, however, 25 mg of the drug was dissolved in 0.8 ml water containing a dispersion of lecithin particles. The lecithin particles used had an effective diameter of 270 nm and polydispersity of 0.295, as determined by dynamic light scattering (Brookhaven 90Plus), and a zeta potential of −43.4 my. The aqueous dispersion containing the drug was then emulsified in 19 ml of ethyl acetate containing surfactant at 303 K. The resulting water-in-ethyl acetate (W/Ac) emulsion was then transferred into 150 ml ethyl acetate, and the precipitated particles were collected by centrifugation, washed with hexane twice to remove any residual lecithin and surfactant, and then dried at room temperature.

### Example 2

### Interfacial Tension

The interfacial tension (γ) between water (saturated with ethyl acetate) and ethyl acetate (saturated with water) with or without surfactant was measured using a pendant drop tensiometer as described elsewhere.(11) Measurements were carried out inside a sealed cuvette at 298 K. The results shown here represent the average of three independent measurements, with a deviation of 0.05 mN·m⁻¹ or less. Since no experimental density values of the mutually saturated phases are available in the literature, the density of pure water and ethyl acetate were used to calculate γ. While this assumption will certainly impact the actual tension values, the relative trend with the various surfactants should hold, as the surfactant concentration is very small and should not affect the relative solubility of the phases, and thus the baseline density.

### Example 3

### Particle Characterization

The particle size and morphology were confirmed by scanning electron microscopy (SEM, Hitachi S-2400). Several drops of a suspension of the particle in HPFP were placed on a cover glass slip and allowed to dry. The cover glass substrates were then sputtered for 30 s with gold for SEM analysis at the acceleration voltage of 20 KV. The drug content (SS and THS) in the porous particles was analyzed by UV-vis. A certain amount of SS porous particles was dissolved in 0.1 M NaOH methanol solution. The residual lecithin was removed by centrifugation and the drug concentration in the supernatant was quantified using UV spectroscopy at the detection wavelength of 246 nm.(24) The drug content of porous THS particles was also determined by UV-vis. A known amount of drug was dissolved in methanol, and the absorption was determined at 280 nm.(35) Residual AOT in the porous particles was detected with UV-vis at 216 nm in 0.1 M NaOH methanol solution.

### Example 4

### Physical Stability in Propellant HFAs

An exact mass of the drug particles were initially fed into pressure proof glass vials (68000318, West Pharmarceutical Services), and crimp-sealed with 50 μl metering valves (EPDM Spraymiser™, 3M Inc). Subsequently, a known amount of HFA227 or HFA134a was added with the help of a manual syringe pump (HiP 50-6-15) and a home-built high pressure aerosol filler, to a final drug concentration of 2 mg·ml⁻¹. The dispersions were then sonicated in a low energy sonication bath (VWR, P250D, set to 180 W) in order to break up any large aggregates. The physical stability of the suspensions in HFA was investigated by visually monitoring the dispersion as a function of the time elapsed after mechanical energy input ceased.

### Example 5

### Aerosol Performance Evaluation

The aerosol properties of the formulations containing the solid and porous SS and THS porous particles were determined with an Andersen Cascade Impactor (ACI, CroPharm, Inc.) operated at a flow rate of 28.3 L·min⁻¹. The experiments were carried out at 298 K and 45% relative humidity. Before each test, several shots were first fired to waste. Subsequently, 10 shots were released into the impactor, with an interval of 30 between actuations. Three independent canisters were tested for each formulation. The average and standard deviation from those three independent runs are reported here. The amount of SS and THS deposited on the valve stem, actuator, induction port and stages was collected by rinsing those parts thoroughly with a known volume of 0.1 M NaOH methanol solution (SS) or pure methanol (THS), respectively.

The drug content was then quantified by UV-Vis spectroscopy, with a detection wavelength of 246 nm for SS and 280 nm for THS. The effect of a spacer (Aerochamber Plus) on the aerosol characteristics of the SS formulation was also investigated. The results obtained with the SS formulations proposed here were also contrasted with those obtained with spherical solid SS particles, and a commercial SS formulation (Ventolin® HFA). The same actuator as that of Ventolin® HFA was used in all experiments. The fine particle fraction (FPF) reported in this example is defined as the percentage of drug on the respirable stages of the impactor (stage 3 to terminal filter) over the total amount of drug released into the device (from the induction port to filter).

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods disclosed herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are disclosed herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically disclosed herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or and consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

### CITED REFERENCES

1. J. Bell and S. Newman. The rejuvenated pressurized metered dose inhaler. Expert Opin Drug Discovery. 4:215-234 (2007).
2. R. N. Dalby, S. L. Tiano, and A. J. Hickey. Medical devices for the delivery of therapeutic aerosols to the lungs. Inhalation Aerosols: Physical and Biological Basis for Therapy (2nd Edition), 2007, pp. 417-444.
3. H. D. C. Smyth. Propellant-driven metered-dose inhalers for pulmonary drug delivery Expert Opin Drug Delivery. 2:53-74 (2005).
4. H. M. Courrier, N. Butz, and T. F. Vandamme. Pulmonary drug delivery system: recent developments and prospects. Crit Rev Ther Drug Carrier Syst. 19:425-498 (2002).
5. P. Rogueda. Novel hydrofluoroalkane suspension formulations for respiratory drug delivery. Expert Opin Drug Deliv. 2:625-638 (2005).
6. Tsi-Zong Tzou, R. R. Pachuta, R. B. Coy, and R. K. Schultz. Drug from selection in Albuterol-Containing Metered-Dose inhaler formulation and its impact on chemical and physical stability. J Pharm Sci. 86:1352-1357 (1997).
7. L. Wu, R. P. S. Peguin, P. Selvam, U. Chokshi, and S. R. P. da Rocha. Molecular Scale Behavior in Alternative Propellant-Based Inhaler Formulations. Inhalation Aerosols: Physical and Biological Basis for Therapy (2nd Edition), 2007, pp. 373-397.
8. N. Patel, M. Marlow, and M. J. Lawrence. Formation of fluorinated nonionic surfactant microemulsions in hydrofluorocarbon 134a (HFC 134a). J Colloid Interface Sci. 258:345-353 (2003).
9. N. Patel, M. Marlow, and M. J. Lawrence. Fluorinated ionic surfactant microemulsions in hydrofluorocarbon 134a (HFC 134a). J Colloid Interface Sci. 258:354-362 (2003).
10. P. Selvam, B. Bharatwaj, and S. R. P. da Rocha. Reverse Aqueous Aggregates in Hydrofluoroalkanes for the Non-invasive Regional Delivery of Anti-cancer Therapeutics to the Lungs. Pharm Res. To be submitted: (2008).
11. P. Selvam, U. Chokshi, A. Gouch, L. Wu, L. Porcar, and S. R. P. da Rocha. Ethoxylated copolymer surfactants for the HFA134a-water interface: interfacial activity, aggregate microstructure and biomolecule uptake. Soft Matter 4:357-366 (2008).
12. P. Selvam, R. P. S. Peguin, U. Chokshi, and S. R. P. da Rocha. Surfactant Design for the 1,1,1,2-Tetrafluoroethane-Water Interface: ab initio Calculations and in situ High-Pressure Tensiometry. Langmuir 22:8675-8683 (2006).
13. D. C. Steytler, M. Thorpe, J. Eastoe, A. Dupont, and R. K. Heenan. Microemulsion Formation in 1,1,1,2-Tetrafluoroethane (R134a). Langmuir. 19:8715-8720 (2003).
14. Montreal protocol on substance that deplete the ozone layer. in 261LM 1541 (1987).
15. K. J. McDonald and G. P. Martin. Transition to CFC-free metered dose inhalers—into the new millennium. Int J Pharm. 201:89-107 (2000).
16. C. Vervaet and P. R. Byron. Drug-surfactant-propellant interactions in HFA-formulations. Int J Pharm. 186:13-30 (1999).
17. L. Wu and S. R. P. da Rocha. Biocompatible and Biodegradable Copolymer Stabilizers for Hydrofluoroalkane Dispersions: A Colloidal Probe Microscopy Investigation. Langmuir. 23:12104-12110 (2007).
18. J. S. Stefely, D. W. Schultz, L. E. Schallinger, C. A. Perman, C. L. Leach, and D. C. Duan. Biocompatible compounds for pharmaceutical drug delivery systems. U.S. Pat. No. 6,126,919 (2000).
19. J. S. Stefely, D. C. Duan, P. B. Myrdal, D. L. Ross, D. W. Schultz, and C. L. Leach. Design and utility of a novel class of biocompatible excipients for HFA-based MDIs. Respir Drug Delivery VII:83-90 (2000).
20. L. Wu, R. P. S. Peguin, and S. R. P. da Rocha. Understanding Solvation in Hydrofluoroalkanes: Ab initio Calculations and Chemical Force Microscopy. J Phys Chem B 111:8096-8104 (2007).
21. R. P. S. Peguin, L. Wu, and S. R. P. da Rocha. The Ester Group: How Hydrofluoroalkane-philic is it. Langmuir. 23 8291-8294 (2007).
22. R. P. S. Peguin, P. Selvam, and S. R. P. da Rocha. Microscopic and Thermodynamic Properties of the HFA134a-Water Interface: Atomistic Computer Simulations and Tensiometry under Pressure. Langmuir 22:8826-8830 (2006).
23. R. P. S. Peguin and S. R. P. da Rocha. Solvent-Solute Interactions in Hydrofluoroalkane Propellants. J Phys Chem C (In press) (2008).
24. L. Wu, B. Bharatwaj, J. Panyam, and S. R. P. da Rocha. Core-shell Particles for the Dispersion of Small Polar Drugs and Biomolecules in Hydrofluoroalkane Propellants. Pharm Res. 25:289-301 (2008).
25. L. Wu and S. R. P. da Rocha. Novel Propellant-driven Inhalation Formulations: Engineering Polar Drug Particles with Surface-trapped Hydrofluoroalkane-philes. Eur J Pharm Sci. 33:146-158 (2008).
26. S. A. Jones, G. P. Martin, and M. B. Brown. Stabilization of deoxyribonuclease in hydrofluoroalkanes using miscible vinyl polymers. J Controlled Release. 115:1-8 (2006).
27. Y.-H. Liao, M. B. Brown, S. A. Jones, T. Nazir, and G. P. Martin. The effects of polyvinyl alcohol on the in vitro stability and delivery of spray-dried protein particles from surfactant-free HFA 134a-based pressurised metered dose inhalers. Int J Pharm. 304:29-39 (2005).
28. L. A. Dellamary, T. E. Tarara, D. J. Smith, C. H. Woelk, A. Adractas, M. L. Costello, H. Gill, and J. G. Weers. Hollow porous particles in metered dose inhalers. Pharm Res. 17:168-174 (2000).
29. D. A. Edwards, A. Ben-Jebria, and R. Langer. Recent advances in pulmonary drug delivery using large, porous inhaled particles. J Appl Physiol. 85:379-385 (1998).
30. D. A. Edwards, J. Hanes, G. Caponetti, J. Hrkach, A. Ben-Jebria, M. L. Eskew, J. Mintzes, D. Deaver, N. Lotan, and R. Langer. Large porous particles for pulmonary drug delivery. Science. 276:1868-1871 (1997).
31. R. Vanbever, J. D. Mintzes, J. Wang, J. Nice, D. Chen, R. Batycky, R. Langer, and D. A. Edwards. Formulation and physical characterization of large porous particles for inhalation. Pharm Res. 16:1735-1742 (1999).
32. C. Dunbar, G. Scheuch, K. Sommerer, M. DeLong, A. Verma, and R. Batycky. In vitro and in vivo dose delivery characteristics of large porous particles for inhalation. Inter J Pharm. 245:179-189 (2002).
33. L. A. Dellamary, T. E. Tarara, D. J. Smith, C. H. Woelk, A. Adractas, M. L. Costello, H. Gill, and J. G. Weers. Hollow porous particles in metered dose inhalers. Pharm Res. 17:168-174 (2000).
34. L. Wu and S. R. P. da Rocha. Application of the atomic force microscope in the development of propellant-based inhalation formulation. KONA Powder and Particle Journal. In press: (2008).
35. S. Selma, S. Handan, P. Gilles, E. M. T, S. Mustafa, H. A. Atilla, and K. H. Suheyla. Preparation, characterization and in vivo distribution of terbutaline sulfate loaded albumin microspheres J controlled release. 82:245-258 (2002).

1. A method of preparing a porous particle comprising dissolving at least one water soluble therapeutic in an aqueous dispersion containing nanoparticles;
emulsifying the aqueous dispersion in an organic solvent containing a surfactant;
diluting the emulsion in a larger volume of organic solvent; and
removing trapped nanoparticles.

2. The method of claim 1, wherein the nanoparticles are negatively charged lecithin particles, stabilized by an ionic surfactant adsorbed at the oil-water interface.

3. The method of claim 2, wherein said removing of said trapped nanoparticles is by washing.

4. The method of claim 3, wherein said washing occurs with an interfacially active species having the same charge as the lecithin particles.

5. (canceled)

6. (canceled)
